(19) United States
(12) Patent Application Publication
(10) Pub. No.: US 2023/0061332 A1
(43) Pub. Date: Mar. 2, 2023

(54) METHOD FOR CO-PRODUCTION OF DECARBONIZED METHANOL AND AMMONIA

(71) Applicant: L'Air Liquide, Société Anonyme pour Etude et Exploitation des Procaédés Georges Claude, Paris (FR)

(72) Inventor: Joseph T. STROFFOLINO, IV, Singapore (SG)

(21) Appl. No.: 17/896,377

(22) Filed: Aug. 26, 2022

Related U.S. Application Data
(60) Provisional application No. 63/237,615, filed on Aug. 27, 2021.

(51) Int. Cl. C01C 1/04; C01B 3/50; C07C 29/151

(52) U.S. Cl. CPC C01C 1/0488; C01B 3/508; C07C 29/1518; C01B 2210/0012

(57) ABSTRACT

A process for the co-production of low carbon methanol and ammonia, including providing renewable power to an electrolysis unit, thereby producing at least a green oxygen stream and a green hydrogen stream. Providing renewable power to a nitrogen generation unit, thereby producing a green nitrogen stream. Providing a hydrocarbon stream and some amount of steam to an oxygen based reformer, along with at least part of the green oxygen stream, thereby producing a raw syngas stream. Combining the green hydrogen stream with the oxygen-based reformer feed to adjust the composition of the raw syngas stream to be suitable for methanol synthesis. Introducing the raw syngas stream into either a conventional or once-through methanol synthesis reactor, thereby producing at least a low carbon methanol product (after purification) and an unreacted syngas stream.

### CROSS REFERENCE TO RELATED APPLICATIONS

This application claims the benefit of priority under 35 U.S.C. § 119 (a) and (b) to U.S. Provisional Patent Application No. 63/237,615, filed Aug. 27, 2021, the entire contents of which are incorporated herein by reference.

### BACKGROUND

In recent years, increasing attention has been paid to the impact of carbon dioxide emissions from industrial processes. Particular attention has been paid to the synthesis of ammonia and methanol. There is a need within the industry for an efficient and cost-effective process for the co-production of decarbonized methanol and ammonia.

### SUMMARY

A process for the co-production of low-carbon methanol and ammonia, including providing renewable power to an electrolysis unit, thereby producing at least a green oxygen stream and a green hydrogen stream. Providing renewable power to a nitrogen generation unit, thereby producing a green nitrogen stream. Providing a hydrocarbon stream and some amount of stream to an oxygen-based reformer such as an autothermal reforming unit or a partial oxidation unit, along with at least some part of the green oxygen stream, thereby producing a low-carbon raw syngas stream. Introducing the low-carbon raw syngas stream, without any water gas shift units nor carbon dioxide capture units, into a pressure swing adsorption unit, thereby producing a low-carbon hydrogen stream and a PSA off-gas stream containing all of the produced carbon dioxide as well as hydrogen, carbon monoxide, and other components. Combining the low-carbon hydrogen stream with the green nitrogen stream, thereby producing a low-carbon ammonia synthesis feed stream. Introducing the low-carbon ammonia synthesis feed stream into an ammonia synthesis reactor, thereby producing at least a low-carbon ammonia product. Combining the green hydrogen stream with the PSA off-gas stream, thereby producing a low-carbon methanol synthesis feed stream and introducing the low-carbon methanol synthesis feed stream into a methanol synthesis reactor, thereby producing at least a low-carbon methanol product.

A process for the co-production of low carbon methanol and ammonia, including providing renewable power to an electrolysis unit, thereby producing at least a green oxygen stream and a green hydrogen stream. Providing renewable power to a nitrogen generation unit, thereby producing a green nitrogen stream. Providing a hydrocarbon stream and some amount of steam to an oxygen based reformer, along with at least part of the green oxygen stream, thereby producing a raw syngas stream. Combining the green hydrogen stream with the oxygen-based reformer feed to adjust the composition of the raw syngas stream to be suitable for methanol synthesis. Introducing the raw syngas stream into a once-through methanol synthesis reactor, thereby producing at least a low carbon methanol product (after purification) and an unreacted syngas stream. Introducing the unreacted syngas stream into a pressure swing adsorption unit, thereby producing a pure hydrogen stream and a PSA off-gas stream. Compressing at least a portion of the PSA off-gas stream to be combined with the oxygen-based reformer hydrocarbon feed, or optionally to the methanol reactor inlet. Combining the pure hydrogen stream with the green nitrogen stream, thereby producing a low carbon ammonia synthesis feed stream. Introducing the low carbon ammonia synthesis feed stream into ammonia synthesis reactor(s), thereby producing at least a low carbon ammonia product. Wherein the unreacted syngas stream leaving the methanol reactor is not subjected to a water gas shift conversion prior to introduction into the pressure swing adsorption unit.

A process for the co-production of low carbon methanol and ammonia, including providing renewable power to an electrolysis unit, thereby producing at least a green oxygen stream and a green hydrogen stream. Providing renewable power to a nitrogen generation unit, thereby producing a green nitrogen stream. Providing a hydrocarbon stream and some amount of steam to an oxygen based reformer, along with at least part of the green oxygen stream, thereby producing a raw syngas stream.

During a first operating mode, combining the green hydrogen stream with the oxygen-based reformer feed to adjust the composition of the raw syngas stream to be suitable for methanol synthesis. Introducing the raw syngas stream into a once-through methanol synthesis reactor, thereby producing at least a low carbon methanol product and an unreacted syngas stream. Introducing the unreacted syngas stream into a pressure swing adsorption unit, thereby producing a pure hydrogen stream and a PSA off-gas stream. Compressing at least a portion of the PSA off-gas stream to be combined with the oxygen-based reformer hydrocarbon feed, or optionally to the methanol reactor inlet. Combining the pure hydrogen stream with the green nitrogen stream, thereby producing a low carbon ammonia synthesis feed stream. Introducing the low carbon ammonia synthesis feed stream into ammonia synthesis reactor(s), thereby producing at least a low carbon ammonia product. Wherein the unreacted syngas stream leaving the methanol reactor is not subjected to a water.

During a second operating mode, combining the green hydrogen stream with the hydrocarbon stream or optionally the raw syngas stream, thereby producing a methanol synthesis feed stream. Introducing the methanol synthesis feed stream into a once-through methanol synthesis reactor, thereby producing at least a low carbon methanol product, which is less than the amount produced in the first operating mode, and an unreacted syngas stream. Introducing at least a portion of the unreacted methanol stream into a water-gas shift conversion unit, thereby producing a shifted syngas stream. Introducing the shifted syngas stream into a pressure swing adsorption unit, thereby producing a hydrogen stream and a PSA off-gas stream. Combining the hydrogen stream with the green nitrogen stream, thereby producing an ammonia synthesis feed stream. And Introducing the ammonia synthesis feed stream into an ammonia synthesis reactor, thereby producing at least a low carbon ammonia product, which is greater than the amount produced in the first operating mode.

### BRIEF DESCRIPTION OF THE FIGURES

For a further understanding of the nature and objects for the present invention, reference should be made to the following detailed description, taken in conjunction with the accompanying drawings, in which like elements are given the same or analogous reference numbers and wherein:

FIG. 1 is a schematic representation of a system with a recycle methanol reactor, in accordance with one embodiment of the present invention.

FIG. 2 is a schematic representation of a system with a once-through methanol reactor, in accordance with one embodiment of the present invention.

### ELEMENT NUMBERS

101=hydrocarbon feed stream
102=feed compressor (optional)
103=high-pressure hydrocarbon feed stream
104=oxygen-based reformer
105=raw syngas stream
106=pressure swing adsorption unit (PSA)
107=pure hydrogen stream
108=carbon monoxide removal unit (optional)
109=carbon monoxide lean hydrogen stream (optional)
110=ammonia syngas compressor
111=high-pressure ammonia syngas stream
112=ammonia synthesis reactor(s)
113=ammonia stream
114=ammonia refrigeration unit
115=refrigerated ammonia stream
116=ammonia storage unit(s)
117=ammonia product stream
118=renewable power source(s)
119=electrolyzer
120=green hydrogen stream
121=green oxygen stream
122=gaseous oxygen vent stream
123=remainder stream (of green oxygen stream)
124=gaseous oxygen compressor
125=pure oxygen stream
126=green hydrogen feed stream
127=green hydrogen fuel stream (optional)
128=fired heater
129=carbon dioxide free flue gas stream
130=hydrogen compressor (optional)
131=compressed hydrogen stream
132=first portion (of compressed hydrogen stream)
133=second portion (of compressed hydrogen stream)
134=PSA off-gas stream
135=imported carbon dioxide stream (optional)
136=PSA tail gas compressor inlet stream
137=PSA tail gas compressor
138=compressed tail gas stream
139=methanol synthesis feed stream
140=methanol synthesis reactor
141=raw methanol stream
142=methanol recycle stream
143=methanol recycle compressor inlet stream
144=methanol recycle compressor
145=compressed methanol recycle stream
146=methanol purge gas stream
147=methanol purification
148=methanol stream
149=wastewater stream
150=saturator stream (optional)
151=methanol storage unit
152=methanol product stream
153=green hydrogen export stream (optional)
154=nitrogen generator
155=nitrogen stream
156=ammonia purge gas stream
157=hydrogen fuel stream (optional)
158=water gas shift converter(s) (optional)
159=shifted syngas stream
160=air separation unit (optional)
161=oxygen (optional)
162=nitrogen (optional—replaces 154)
163=saturator (optional)
164=saturated hydrocarbon feed
201=hydrocarbon feed stream
202=feed compressor (optional)
203=high-pressure hydrocarbon feed stream
204=oxygen-based reformer
205=raw syngas stream
206=pressure swing adsorption unit (PSA)
207=pure hydrogen stream
208=ammonia syngas compressor
209=high-pressure ammonia syngas stream
210=ammonia synthesis reactor(s)
211=ammonia stream
212=ammonia refrigeration unit
213=refrigerated ammonia stream
214=ammonia storage unit
215=ammonia product stream
216=renewable power source
217=electrolysis unit
218=green hydrogen stream
219=green oxygen stream
220=gaseous oxygen vent stream
221=remainder stream (of green oxygen stream)
222=gaseous oxygen compressor
223=pure oxygen stream to oxygen-based reformer
224=fired heater
225=low carbon dioxide flue gas stream
226=hydrogen compressor (optional)
227=compressed hydrogen stream
228=PSA off-gas stream
229=methanol synthesis feed stream
230=once-through methanol synthesis reactor
231=raw methanol stream
232=methanol purification
233=methanol stream
234=wastewater stream
235=saturator stream (optional—not shown)
236=methanol storage unit
237=methanol product stream
238=green hydrogen export stream (optional)
239=nitrogen generator
240=nitrogen stream
241=ammonia purge gas stream
242=hydrogen purge stream
243=water gas shift converter (optional)
244=shifted syngas stream (optional)
245=recycle stream to methanol reactor (optional)
246=compressed tail gas stream
247=oxygen based reformer feed stream
248=compressed hydrogen stream
249=combined methanol feed stream
250=unreacted syngas stream
251=first portion (of PSA off-gas stream)
252=second portion (of PSA off-gas stream)
253=ammonia reactor(s) feed stream
254=PSA off-gas compressor
255=a portion of compressed hydrogen stream
256=air separation unit (optional)
257=oxygen stream
258=nitrogen stream

### DESCRIPTION OF PREFERRED EMBODIMENTS

Illustrative embodiments of the invention are described below. While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

As used herein, the term “carbon footprint” is defined as kg of carbon dioxide emitted divided by the standard chemical energy values for the hydrogen, ammonia and methanol product streams.

As used herein, the terms “green oxygen” and “green hydrogen” are defined as having been produced entirely by the use of renewable energy.

As used herein, the terms “low-carbon ammonia”, “low-carbon methanol”, and “low-carbon hydrogen” are defined as having lower “carbon footprints” than conventional production means.

As used herein, the term “once through methanol reactor” is defined as a single pass reactor that operates without the direct recirculation of unconverted synthesis gas. It may still receive indirect recirculation of unconverted carbon oxides and methane, but not the majority of unconverted hydrogen, which is removed after the reactor.

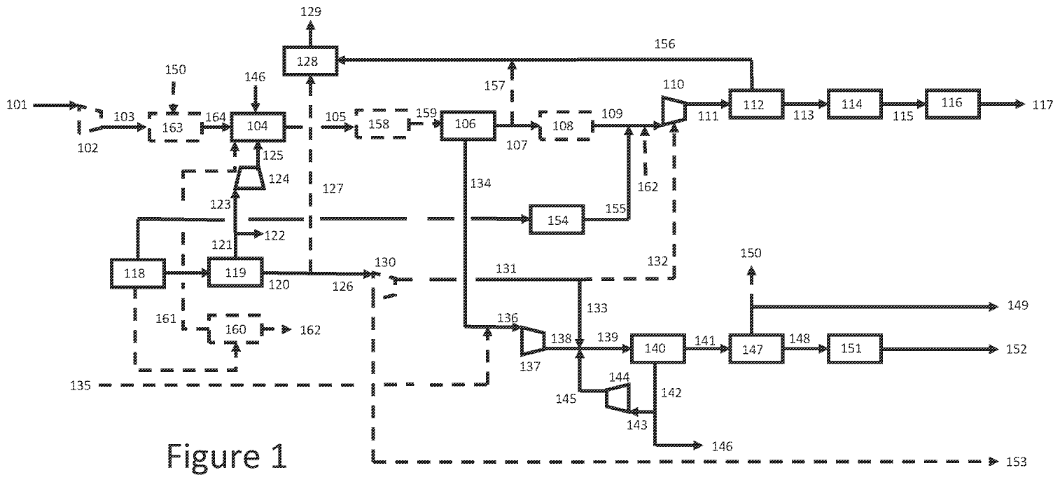

Turning to FIG. 1, a method for co-producing low-carbon ammonia and methanol is provided. Renewable power supply 118 may be any traditional renewable power source such as, for example, hydro-electric power, wind power, or solar power, or a mix of such sources. Renewable power supply 118 provides power to, at least, nitrogen generator 154 and electrolysis unit 119. The nitrogen generator 154 produces high purity nitrogen stream 155. Through the use of renewable power, electrolysis unit 119 produces green oxygen stream 121 and green hydrogen stream 120. A portion of green oxygen stream 121 may be vented as gaseous oxygen vent stream 122 if the full amount of stream 121 is not required for the oxygen-based reforming. The remainder 123 of green oxygen stream 121 has its pressure increased in gaseous oxygen compressor 124, thereby producing pure oxygen stream 125.

Optionally, the nitrogen generator may be replaced by an air separation unit 160, whereby air separation unit 160 produces a nitrogen stream 162 (to replace stream 155) and an oxygen stream 161 (to replace or supplement stream 125).

Hydrocarbon feed stream 101 (either free of catalyst poisons, or with catalyst poisons removed—not shown, or if oxygen-based reformer 104 is non catalytic, removed later—not shown) is introduced into oxygen-based reformer 104. Oxygen-based reformer 104 may be an autothermal reformer (ATR) or partial oxidation (POX). Steam addition (not shown) or use of a saturator 104 may also be performed. Hydrocarbon feed stream 101 may be increased in pressure in optional feed compressor 102, thereby producing high-pressure hydrocarbon feed stream 103. Oxygen-based reformer 104 is also fed with pure oxygen stream 125, thereby producing raw syngas stream 105. Raw syngas stream 105 comprises hydrogen, carbon monoxide, carbon dioxide, residual methane and possibly traces of nitrogen. In one embodiment, raw syngas stream 105 is not subjected to a water gas shift or carbon capture unit. In another embodiment, raw syngas stream 105 may be introduced into optional water gas shift converter(s) 158, wherein it is partially or fully shifted thereby producing hydrogen enriched shifted syngas stream 159 but is still not subject to a carbon capture unit.

Raw syngas stream 105 is introduced into pressure swing adsorption unit (PSA) 106. Raw syngas stream 105 may be cooled as necessary (not shown) prior to introduction into PSA 106. Raw syngas stream 105 is not subjected to a water gas shift reaction at any point, nor is it subjected to any carbon capture technology.

PSA 106 produces at least pure hydrogen stream 107 and PSA off-gas stream 134. PSA 106 has a hydrogen recovery rate of greater than 75%. If the pressure swing adsorption unit 106 has a recovery that is below 75%, less hydrogen will be present in pure hydrogen stream 107 and more hydrogen will be present in PSA off-gas stream 134. In this situation, the additional hydrogen in PSA off-gas stream 134 offsets a portion of the green hydrogen in second portion 133 that is used in the methanol synthesis. In order to make up for the loss of hydrogen in pure hydrogens stream 107, additional green hydrogen can be introduced through first portion 132.

PSA off-gas stream 134 comprises hydrogen, carbon monoxide, carbon dioxide, residual methane and possibly traces of nitrogen. Optionally, PSA off-gas stream 134 may be combined with imported carbon dioxide stream 135, thereby forming PSA tail gas compressor inlet stream 136. PSA tail gas compressor inlet stream 136 (or, alternatively, PSA off-gas stream 134) is then introduced into PSA tail gas compressor 137, thereby producing compressed tail gas stream 138.

Pure hydrogen stream 107 may then be introduced into (optional) carbon monoxide removal unit 108, in order to remove at least a part of any carbon monoxide that may be present. Carbon monoxide removal unit 108 may be a carbon monoxide adsorber or a methanation unit. Resulting carbon monoxide lean hydrogen stream 109 (or, alternatively, pure hydrogens stream 107) is then combined with high-pressure nitrogen stream 155, and the combined stream is introduced into ammonia feed compressor 110.

Ammonia feed compressor 110 produces high-pressure ammonia feed stream 111, which is then introduced into ammonia synthesis reactor 112. Ammonia synthesis reactor 112 produces ammonia stream 113, which is introduced into ammonia refrigeration unit 114. Ammonia refrigeration unit 114 produces refrigerated ammonia stream 115, which is then sent to ammonia storage unit 116. As required by external demand, ammonia product stream 117 is removed from ammonia storage unit 116.

Green hydrogen stream 120 is divided into at least green hydrogen feed stream 126 and green hydrogen fuelstream 127. Green hydrogen fuelstream 127 is introduced into fired heater 128 and the resulting low carbon dioxide flue gas stream 129 is vented from the system. Fired heater 128 provides preheating and superheating within the system (not shown), and is fueled by ammonia purge gas stream 156, hydrogen 157, methanol purge gas 163, and/or green hydrogen fuel 127, and thus provides minimal carbon dioxide emissions. Green hydrogen feed stream 126 may be introduced into (optional) hydrogen compressor 130, thereby producing compressed hydrogen stream 131. Optionally electrolysis unit 119 may be over sized such that additional green hydrogen may exit the system as product green hydrogen export stream 153. Compressed hydrogen stream 131 (or, alternatively, green hydrogen feed stream 126) may then be divided into at least two portions. The first portion 132 may optionally be introduced into ammonia feed compressor 110. The second portion 133 is combined with compressed tail-gas stream 138 and compressed methanol recycle stream 145, thereby forming methanol synthesis feed stream 139.

Methanol synthesis feed stream 139 is introduced into methanol synthesis reactor 140, thereby producing raw methanol stream 141, and methanol recycle stream 142. Methanol recycle stream 142 is divided into methanol recycle compressor inlet stream 143 and methanol purge stream 146. Methanol purge stream 146 may be recycled back to the oxygen-based reformer 104 (not shown) or in part, the fired heater 128 (not shown). Methanol recycle compressor inlet stream 143 is introduced into methanol recycle compressor 144, thereby producing compressed methanol recycle stream 145. Raw methanol stream 141 is introduced into methanol distillation column 147, thereby producing at least methanol stream 148 and wastewater stream 149. Second portion 133 is green hydrogen, thus methanol stream 148 is blue methanol. Wastewater stream may be divided into saturator stream 150, which is returned to the oxygen-based reformer 104 by means of a saturator 163, with the remainder being sent to a water reclamation system (not shown). Methanol stream 148 is then sent to methanol storage unit 151. As required by external demand, methanol product stream 152 is removed from methanol storage unit 151.

This system results in a very low-carbon footprint. At least 95% of the carbon that is introduced to the process (via stream 101), is leaving the system as methanol (via stream 152), and not as carbon dioxide (via stream 129), without use of any carbon dioxide capture or storage methods.

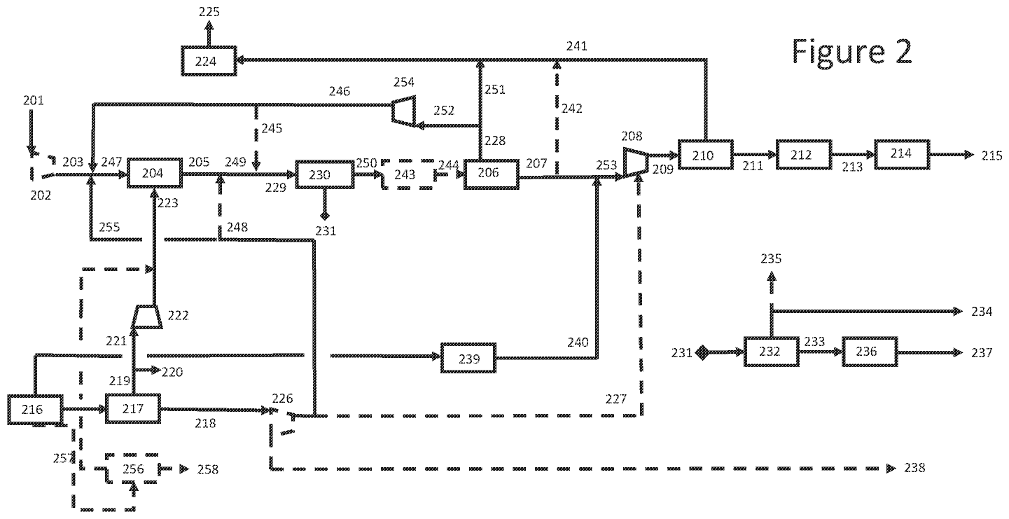

Turning to FIG. 2, a method for co-producing low carbon ammonia and methanol utilizing once-through methanol synthesis reactor 230 is provided. Renewable power supply 216 may be any traditional renewable power source such as, for example, hydro-electric power, wind power, or solar power, or a mix thereof. Renewable power supply 216 provides power to, at least, a nitrogen generator 239 and electrolysis unit 217. The nitrogen generator 239 produces nitrogen stream 240. Through the use of renewable power, electrolysis unit 217 produces green oxygen stream 219 and green hydrogen stream 218. A portion of green oxygen stream 219 may be vented as gaseous oxygen vent stream 220 if the full amount of stream 219 is not required for the oxygen-based reforming. The remainder 221 of green oxygen stream 219 has its pressure increased in gaseous oxygen compressor 222, thereby producing pure oxygen stream 223.

Optionally, the nitrogen generator 239 may be replaced by an air separation unit 256, whereby air separation unit 256 produces a nitrogen stream 258 (to replace stream 240) and an oxygen stream 257 (to replace or supplement stream 223).

Green hydrogen stream 218 may be compressed in optional hydrogen compressor 226 as needed by the system, thereby producing compressed hydrogen stream 248. Compressed hydrogen stream 218 is combined with raw syngas stream 205, thereby producing combined methanol feed stream 249. A portion 227 of compressed hydrogen stream 218 may be introduced into ammonia feed compressor 210. A portion of compressed hydrogen stream 218 may exit the system as green hydrogen export stream 238.

Hydrocarbon feed stream 201 may be increased in pressure in optional feed compressor 202, thereby producing high-pressure hydrocarbon feed stream 203 which is combined with compressed tail gas stream 246, and at least a portion 255 of compressed hydrogen stream 248, and combined oxygen-based reformer feed stream 247 is introduced into an oxygen based reformer such as an autothermal reformer (ATR) or partial oxidation (POX) 204. Oxygen based reformer 204 is also fed with pure oxygen stream 223, thereby producing raw syngas stream 205. Raw syngas stream 205 comprises hydrogen, carbon monoxide, carbon dioxide, residual methane and possibly traces of nitrogen. Raw syngas stream 205 is optionally combined with compressed hydrogen stream 248, and optionally compressed tail gas stream 245, thereby forming methanol synthesis reactor feed stream 229. Compressed hydrogen feed 255 and optionally 248 are used to balance any hydrogen deficiencies in methanol reactor feed stream 229.

Methanol synthesis feed stream 229 is introduced into once-through methanol synthesis reactor 240, thereby producing raw methanol stream 231, and unreacted syngas stream 250. Raw methanol stream 231 is introduced into methanol distillation column 232, thereby producing at least methanol stream 233 and wastewater stream 234. Wastewater stream 234 may be divided into saturator stream 235, which is returned to the ATR by means of a saturator (not shown), with the remainder being sent to a water reclamation system (not shown). Methanol stream 233 is then sent to methanol storage unit 236. As required by external demand, methanol product stream 237 is removed from methanol storage unit 236. Unreacted syngas stream 250 is optionally introduced into water gas shift converter(s) 243 wherein it may be partially or fully shifted thereby producing hydrogen enriched shifted syngas stream 244. Stream 250 (or in the optional case of shift, Stream 244) is introduced into pressure swing adsorption unit (PSA) 206. In the present system, unreacted syngas stream 250 is not shifted and directly purified in 206.

PSA 206 produces at least pure hydrogen stream 207 and PSA off-gas stream 228. PSA 206 has a hydrogen recovery rate of greater than 75%. PSA off-gas stream 228 comprises hydrogen, carbon monoxide, carbon dioxide, residual methane and possibly traces of nitrogen.

PSA off-gas stream 228 may be divided into at least two portions. First portion 251 may be sent to fired heater 224 as fuel to control inerts. Second portion 252 may be compressed in PSA tail gas compressor 254, thereby producing compressed tail gas stream 246. Compressed tail gas stream 246 may be combined with hydrocarbon feed stream 203, or a portion 245 may be combined with raw syngas stream 205 or hydrocarbon feed stream 203 and compressed hydrogen stream 248, as discussed above. This routing creates an indirect recirculation of unconverted carbon oxides and methane, but not a majority of unconverted hydrogen, which is produced by the PSA 206 as pure hydrogen stream 207.

Pure hydrogens stream 207 is combined with the nitrogen stream 240 (or optionally the air separation unit nitrogen stream 258), and combined ammonia reactor feed stream 253 is introduced into ammonia feed compressor 208. Pure hydrogens stream 207 is low carbon hydrogen, and nitrogen stream 240 is green nitrogen. Optionally, a portion 242 of pure hydrogen stream 207 may be sent to fired heater 224 as carbon-free fuel.

Ammonia feed compressor 208 produces high-pressure ammonia feed stream 211, which is introduced into ammonia synthesis reactor 210. Ammonia synthesis reactor 210 produces ammonia stream 211, and ammonia purge gas stream 241. Ammonia purge gas stream 241 is introduced into fired heater 224 as fuel. Ammonia stream 211 is introduced into ammonia refrigeration unit 212. Ammonia refrigeration unit 212 produces refrigerated ammonia stream 213, which is then sent to ammonia storage unit 214. As required by external demand, ammonia product stream 215 is removed from ammonia storage unit 214.

This system results in a very low carbon footprint. At least 95% of the carbon that is introduced to the process (via stream 201) is leaving the system as methanol (via stream 237), and not as carbon dioxide (via stream 225), without the use of any carbon dioxide capture or storage methods.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A process for the co-production of low-carbon methanol and ammonia, comprising:
providing renewable power to an electrolysis unit, thereby producing at least a green oxygen stream and a green hydrogen stream,
providing renewable power to a nitrogen generation unit, thereby producing a green nitrogen stream,
providing a hydrocarbon stream and a steam stream to an oxygen-based reformer along with at least a portion of the green oxygen stream, thereby producing a low carbon raw syngas stream,
introducing the low carbon raw syngas stream, without being subjected to a water gas shift process or a carbon dioxide capture process, into a pressure swing adsorption unit, thereby producing a low-carbon hydrogen stream and a PSA off-gas stream,
combining the low-carbon hydrogen stream with the green nitrogen stream, thereby producing a low-carbon ammonia synthesis feed stream,
introducing the low-carbon ammonia synthesis feed stream into an ammonia synthesis reactor, thereby producing at least a low-carbon ammonia product,
combining the green hydrogen stream with the PSA off-gas stream, thereby producing a low-carbon methanol synthesis feed stream, and
introducing the low-carbon methanol synthesis feed stream into a methanol synthesis reactor, thereby producing at least a low-carbon methanol product.

2. The process of claim 1, wherein the low-carbon raw syngas stream may be at least partially treated to a water gas shift process, prior to the pressure swing adsorption unit, thereby producing additional low-carbon hydrogen to the ammonia synthesis feed stream and providing additional green hydrogen to the methanol synthesis feed stream.

3. The process of claim 1, wherein at least a portion of the green hydrogen is combined with additional green nitrogen to produce additional flow to the ammonia synthesis feed stream, thereby generating at least a portion of green ammonia product together with the low-carbon ammonia product.

4. The process of claim 1, further comprising introducing the low-carbon hydrogen stream into a carbon monoxide removal unit prior to introducing the ammonia synthesis feed stream into the ammonia synthesis reactor, as required to protect the ammonia synthesis catalyst.

5. The process of claim 1, wherein the pressure swing adsorption unit has a hydrogen recovery rate of 75% or greater.

6. The process of claim 1, wherein the pressure swing adsorption unit has a hydrogen recovery rate of less than 75%, wherein at least a portion of the green hydrogen stream is introduced into the ammonia synthesis feed stream.

7. The process of claim 1, further comprising a wastewater stream being generated by the methanol synthesis reactor, wherein the wastewater stream is recycled back to the autothermal reformer.

8. The process of claim 1, further comprising importing a carbon dioxide stream, which is then combined with the PSA off-gas stream and the green hydrogen stream, thus forming the methanol synthesis feed stream.

9. A process for the co-production of low carbon methanol and ammonia, comprising:
providing renewable power to an electrolysis unit, thereby producing at least a green oxygen stream and a green hydrogen stream,
providing renewable power to a nitrogen generation unit, thereby producing a green nitrogen stream,
providing a hydrocarbon stream and some amount of steam to an oxygen based reformer along with at least part of the green oxygen stream, thereby producing a raw syngas stream,
combining the green hydrogen stream with the oxygen-based reformer feed to adjust the composition of the raw syngas stream to be suitable for methanol synthesis,
introducing the raw syngas stream into a once-through methanol synthesis reactor, thereby producing at least a low carbon methanol product (after purification) and an unreacted syngas stream,
introducing the unreacted syngas stream into a pressure swing adsorption unit, thereby producing a pure hydrogen stream and a PSA off-gas stream,
compressing at least a portion of the PSA off-gas stream to be combined with the oxygen-based reformer hydrocarbon feed, or to the methanol reactor inlet,
combining the pure hydrogen stream with the green nitrogen stream, thereby producing a low carbon ammonia synthesis feed stream, and
introducing the low carbon ammonia synthesis feed stream into ammonia synthesis reactor(s), thereby producing at least a low carbon ammonia product,
wherein the unreacted syngas stream leaving the methanol reactor is not subjected to a water gas shift conversion prior to introduction into the pressure swing adsorption unit.

10. The process of claim 9, wherein at least a portion of the green hydrogen is combined with additional green nitrogen to produce additional flow to the ammonia synthesis feed stream, thereby generating at least a portion of green ammonia product together with the low carbon ammonia product.

11. The process of claim 9, wherein the pressure swing adsorption unit has a hydrogen recovery rate of 75% or greater.

12. The process of claim 9, wherein the pressure swing adsorption unit has a hydrogen recovery rate of less than 75%, wherein at least a portion of the green hydrogen stream is introduced into the ammonia synthesis feed stream.

13. The process of claim 9, further comprising a wastewater stream being generated by the methanol synthesis reactor, wherein the wastewater stream is recycled back to the oxygen-based reforming via a saturator or as steam.

14. A process for the co-production of low carbon methanol and ammonia, comprising:
providing renewable power to an electrolysis unit, thereby producing at least a green oxygen stream and a green hydrogen stream,
providing renewable power to a nitrogen generation unit, thereby producing a green nitrogen stream, and
providing a hydrocarbon stream and some amount of steam to an oxygen based reformer, along with at least part of the green oxygen stream, thereby producing a raw syngas stream,
in a first operating mode,
combining the green hydrogen stream with the oxygen-based reformer feed to adjust the composition of the raw syngas stream to be suitable for methanol synthesis,
introducing the raw syngas stream into a once-through methanol synthesis reactor, thereby producing at least a low carbon methanol product and an unreacted syngas stream,
introducing the unreacted syngas stream into a pressure swing adsorption unit, thereby producing a pure hydrogen stream and a PSA off-gas stream,
compressing at least a portion of the PSA off-gas stream to be combined with the oxygen-based reformer hydrocarbon feed, or to the methanol reactor inlet,
combining the pure hydrogen stream with the green nitrogen stream, thereby producing a low carbon ammonia synthesis feed stream, and
introducing the low carbon ammonia synthesis feed stream into ammonia synthesis reactor(s), thereby producing at least a low carbon ammonia product,
wherein the unreacted syngas stream leaving the methanol reactor is not subjected to a water,
in a second operating mode,
combining the green hydrogen stream with the hydrocarbon stream or optionally the raw syngas stream, thereby producing a methanol synthesis feed stream,
introducing the methanol synthesis feed stream into a once-through methanol synthesis reactor, thereby producing at least a low carbon methanol product, which is less than the amount produced in the first operating mode, and an unreacted syngas stream,
introducing at least a portion of the unreacted methanol stream into a water-gas shift conversion unit, thereby producing a shifted syngas stream,
introducing the shifted syngas stream into a pressure swing adsorption unit, thereby producing a hydrogen stream and a PSA off-gas stream,
combining the hydrogen stream with the green nitrogen stream, thereby producing an ammonia synthesis feed stream, and
introducing the ammonia synthesis feed stream into an ammonia synthesis reactor, thereby producing at least a low carbon ammonia product, which is greater than the amount produced in the first operating mode.

15. The process of claim 14, wherein during the second operating mode at least a portion of the PSA off-gas stream is compressed and combined with the hydrocarbon feed stream prior to introduction into the oxygen based reformer.

16. The process of claim 14, wherein during the second operating mode at least a portion of the green hydrogen is combined with additional green nitrogen to produce additional flow to the blue ammonia synthesis feed stream.

17. The process of claim 14, wherein during the second operating mode the pressure swing adsorption unit has a hydrogen recovery rate of 75% or greater.

18. The process of claim 14, wherein during the second operating mode the pressure swing adsorption unit has a hydrogen recovery rate of less than 75%, wherein at least a portion of the green hydrogen stream is introduced into the ammonia synthesis feed stream.

19. The process of claim 14, further comprising a wastewater stream being generated by the methanol synthesis reactor, wherein the wastewater stream is recycled back to the oxygen-based reforming via a saturator or as steam.
